(19) United States
(12) Patent Application Publication
(10) Pub. No.: US 2025/0176839 A1
(43) Pub. Date: Jun. 5, 2025

(54) Heart Failure Diagnostic Tools and Methods Using Signal Trace Analysis

(71) Applicant: Foundry Innovation & Research 1, Ltd., Dublin (IE)

(72) Inventors: Friedrich Wetterling, Dublin (IE); Fiachra M. Sweeney, Dublin (IE); Daire Corley-Carmody, Dublin (IE); Stephen Sheridan, Dublin (IE); James Tucker, Dublin (IE); Robert Gaul, Dublin (IE); John R. Britton, Dublin (IE); Teresa Maria Buxo, Dublin (IE); Damien O'Rourke, Dublin (IE)

(21) Appl. No.: 18/845,019

(22) PCT Filed: Mar. 9, 2023
(86) PCT No.: PCT/IB2023/052269

§ 371 Date: Sep. 9, 2024

Related U.S. Application Data
(60) Provisional application No. 63/318,216, filed on Mar. 9, 2022.

(51) Int. Cl. A61B 5/0205

(52) U.S. Cl. CPC A61B 5/0205

(57) ABSTRACT

Systems and methods disclosed offer improved heart failure patient outcomes by automatedly extracting features from vessel area traces and generating cardiac health indicative parameters based on extracted features to provide a normalized congestion index to assess patient status. Examples include diagnostic engine (102) receiving area traces from trace generator (104) and communicating patient status information through user interfaces (116). The diagnostic engine may comprise trace feature detector (106), metrics generator (108), boundary generator (110), index generator (112) and decision logic (114). Disclosed systems may be executed using one or more processing devices or in a networked, distributed processing system.

### RELATED APPLICATION DATA

This application claims the benefit of priority of U.S. Provisional Patent Application Ser. No. 63/318,216, filed Mar. 9, 2022, and titled “Heart Failure Diagnostic Tools and Methods Using Signal Trace Analysis”, which is incorporated by reference herein in its entirety.

### FIELD

The present disclosure relates to heart failure diagnostics and, more particularly, to tools and methods to facilitate use of data produced by heart-failure-related sensors to improve patient outcomes.

### BACKGROUND

Hemodynamic congestion is generally now measured using catheter-based filling pressure of right atrial pressure (RAP) and pulmonary capillary wedge pressure (PCWP). However, due to the small ranges of these pressures relative to the potential measurement error, in the range of 2 to 6 mmHg (RAP), and 4 to 12 mmHg (PCWP), it is challenging to capture relevant pressure changes using common pressure-sensing detectors such as a Swan-Ganz catheter or other implantable pressure sensors, which generally may have accuracy limitations in the range of about ±5 mmHg.

The present Applicant has previously developed and disclosed a number of different sensors for determining patient fluid status based on direct measurement of a vascular dimension, which indicates geometry, namely, cross-sectional area and distension or collapse of the vessel. This measurement of vessels, particularly of the inferior vena cava (IVC), may relate more directly to a patient's circulating blood volume and congestion status. Therefore, such measurements with these sensors could potentially be used to estimate a patient's circulating blood volume and congestion status. In particular, these sensors could potentially be used to determine whether circulating blood volume is too high or too low, whether circulating blood volume is increasing or decreasing and potentially what treatment should be prescribed, such as diuretics or vaso-dilators.

New devices developed and disclosed by the present Applicant include external ultrasound devices as well as implantable sensors capable of long-term placement suitable for monitoring patients with chronic conditions. Examples of such implantable, wireless sensors and external ultrasound devices are disclosed, for example, in U.S. patent application Ser. No. 15/549,042, filed Aug. 4, 2017 (U.S. Pat. No. 10,905,393, granted Feb. 2, 2021), and entitled “Implantable Devices and Related Methods for Heart Failure Monitoring” and U.S. patent application Ser. No. 16/177,183, filed Oct. 31, 2018 (U.S. Pat. No. 10,806,352, granted Oct. 20, 2020) and entitled “Wireless Vascular Monitoring Implants,” each of which is incorporated herein in its entirety. In other clinical situations, such as shorter term acute condition monitoring and in-hospital treatments, vascular dimension sensors for direct fluid state determination and monitoring may be catheter-based. Examples of such catheter-based sensors are disclosed, for example, in U.S. patent application Ser. No. 15/750,100, filed Feb. 2, 2018 (U.S. Pat. No. 11,039,813, granted Jun. 22, 2021) and entitled “Devices and Methods for Measurement of Vena Cava Dimensions, Pressure and Oxygen Saturation,” which is incorporated herein in its entirety.

The present Applicant has also developed and disclosed novel diagnostic and treatment systems and methods based on the use of the aforementioned sensor devices, some of which are disclosed in the above-mentioned patent applications, and further of which are disclosed in U.S. Pat. No. 11,564,596, granted Jan. 31, 2023, entitled “Systems and Methods for Patient Fluid Management,” and U.S. Patent Pub. No. US 2021/0244381 A1, published Aug. 12, 2021, entitled “Patient Fluid Management Systems and Methods Employing Integrated Fluid Status Sensing,” each of which is incorporated by reference herein in its entirety.

Notwithstanding the foregoing advances, challenges remain with respect to clinical interpretation of the signals produced by various vascular area sensing technologies and, in some cases, novel datasets produced thereby.

Among the challenges is calibration of measurements for vessel volume-based parameters arising from intra-individual and inter-individual variations due to heterogeneity of the vessel shape in order to generate a comparable quantitative output metric indicative of right atrial filling pressure or congestion status. For example, the area of the IVC depends on the location observed along the IVC and on the individual, making it difficult to draw comparisons of absolute area measures between patients as a group, which may inhibit clinical use of the data generated. While generally available data normalization algorithms might be applied to address this limitation, when arbitrarily normalizing any feature the normal range will very likely be enclosed by thresholds of unusual and non-intuitive value, i.e. the normalized variable will be in units (possibly dimensionless), with associated thresholds, that are unfamiliar to the clinician and not a part of standard practice and guidelines. Such unusual numbering is a challenge for physicians, and patients, especially as methods evolve and diagnostic algorithms are refined with higher degrees of complexity and the patients increasingly become relevant consumers of the data.

General clinical acceptance of new data types can also present challenges. Long-standing and well-known physiological parameters have their well-understood ranges (such as, blood pressure, body temperature, respiration rate, etc.). These are developed over time, become domain specific knowledge, and become included in clinical guidelines, etc. To help facilitate acceptance and encourage use of new, advantageous systems and data sets, there is thus a need to develop correlations to known physiological parameters for the new signals and datasets to facilitate use by clinicians.

With multiple discrete inputs to diagnostic and treatment algorithms, it is challenging to set actionable thresholds without the instructions becoming overly complex. There is thus a need to reduce the data review burden on the user (whether patient or health care provider) as new datasets and parameters are introduced. Challenges facing the user include definition of action thresholds and also determining which drug from their arsenal to deploy and at what dosage because they often do not have solid inputs to inform drug titration decisions, nor good methods to monitor patient drug adherence, nor good measures of individual responses to different doses of drugs.

Various embodiments disclosed herein address these challenges and present solutions designed to improve speed and accuracy of heart failure diagnostic and treatment decisions and thus improve patient outcomes and reduce hospitalization occurrences and costs.

### SUMMARY

In one implementation, the present disclosure is directed to an automated heart failure diagnostic device. The device includes a trace feature detector to identify selected features and at least one of magnitude and timing of the identified features for received periodic vessel area traces representing changes in fluid state of a patient over time, wherein the selected features comprise one or more of interval time per respiration cycle, area magnitude of respiration modulation, interval time per cardiac cycle, area magnitude of cardiac modulation, dominant cardiac peaks, second cardiac peaks, respiration related area reduction, maneuver types and maximum and minimum areas associated with identified maneuvers; a metrics generator to generate heart function-related parameters for each area trace based on the identified features, magnitudes and timing, the heart function-related parameters comprising one or more of maximum vessel area (Aₘₐₓ), minimum vessel area (Amin), mean vessel area (Aₘₑₐₙ), heart rate (HR), respiration rate (RR), collapsibility index (CI), collapse (C) and cardiac output (CO); a boundary generator to generate at least a vessel lower area boundary (LB) for the patient or a vessel upper area boundary (UB) for the patient using the generated heart function-related parameters; and an index generator to set a patient congestion index based on one or more of the generated heart-function related parameters and at least one of a vessel lower area boundary (LB) or upper area boundary (UB) for the patient, the congestion index indicating patent fluid state on a normalized scale for each the trace period.

In another implementation, the present disclosure is directed to a system automatedly determining patient fluid state using periodic vessel area traces. The system includes a trace feature detector to identify selected features of the vessel area traces and at least one of magnitude and timing of the identified features for the vessel area traces; a metrics generator to generate heart function-related parameters for each area trace based on the identified magnitudes and timing, the heart function-related parameters including at least maximum vessel area (Aₘₐₓ) and minimum vessel area (Aₘᵢₙ); and an index generator to generate a patient congestion index based on at least the maximum vessel area (Aₘₐₓ), the minimum vessel area (Aₘᵢₙ) and at least one of a vessel lower area boundary (LB) or upper area boundary (UB) for the patient, the congestion index indicating patent fluid state on a normalized scale for each the trace period.

In still another implementation, the present disclosure is directed to a computer-based method. The method includes receiving a quiet respiration vessel area trace for a patient within at least one processing device; receiving patent specific information comprising at least patient weight and patient age within the processing device; filtering the quiet respiration vessel area trace at the processing device to identify component signals comprising at least a respiration trace, a cardiac trace, and a mean trace; extracting magnitude and timing features from the traces at the processing device corresponding to at least one or more of area maximum, area minimum, collapse, respiration collapse, cardiac collapse, heart rate and respiration rate; generating at the processing device heart function-related parameters using executable program instructions defining the heart function-related parameters based on the extracted magnitude and timing features, the heart function-related parameters comprising one or more of respiration rate, cardiac output, heart rate, collapsibility index, collapse, maximum vessel area, minimum vessel area and mean vessel area; generating a patient congestion index based on one or more of the magnitude features and heart function-related parameters; and applying weighting to the congestion index, heart function-related parameters and patient specific information using a machine learning based model to return a hospitalization probability score for the patient.

In yet another implementation, the present disclosure is directed to a method for automatedly determining patient fluid state using periodic vessel area traces. The method includes receiving a vessel area trace; identifying selected features and at least one of magnitude and timing of the identified features for the vessel area traces; generating heart function-related parameters for each area trace based on the identified magnitudes and timing, the heart function-related parameters including measured vessel areas and a vessel area boundary; generating a patient congestion index representing a relationship between measured vessel area as determined for an area trace and a vessel area boundary for the patient, the congestion index indicating patent fluid state on a normalized scale for each the trace period.

### BRIEF DESCRIPTION OF DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a high-level schematic depiction of systems encompassed by the present disclosure.

FIG. 2 is an example of an IVC area trace, including a detailed view of a trace portion with some key trace features identified.

FIG. 3 is a block diagram of an embodiment of an overall system according to the present disclosure.

FIG. 4 is a block diagram of an embodiment of a trace generator according to the present disclosure.

FIG. 5 is a block diagram of an embodiment of a feature detector according to the present disclosure.

FIG. 6 is a series of area trace plots showing area response to different patient maneuvers.

FIG. 6A is an enlarged, detailed view of the area trace for a supine breath-hold maneuver as shown in FIG. 6.

FIG. 7 is a block diagram of an embodiment of a metrics generator according to the present disclosure.

FIG. 7A is a block diagram illustrating an embodiment of a component of the metrics generator for extraction of respiration rate from an area trace.

FIG. 7B includes an example of a spectrogram generated from an area trace illustrating another component of the metrics generator in an alternative embodiment for extraction of heart rate and respiration rate from an area trace.

FIG. 7C is an area trace illustrating another component of the metrics generator in an alternative embodiment for determination of area trace features.

FIG. 7D is an area trace illustrating components of the metrics generator in alternative embodiments for determination of collapsibility index.

FIG. 8 is a block diagram of an embodiment of a boundary generator according to the present disclosure.

FIG. 9 shows area trace plots for normal respiration and an inspiration breath-hold maneuver, and illustrates an example of identifying an upper boundary from using a maneuver.

FIG. 10 is a block diagram of an embodiment of an index generator according to the present disclosure.

FIG. 11 is a block diagram of an embodiment of a decision logic according to the present disclosure.

FIG. 12 is a block diagram showing embodiments of interface devices according to the present disclosure.

FIG. 13 is a flow diagram illustrating an embodiment of an overall system flow according to the present disclosure.

FIG. 14 is a block diagram illustrating components of an exemplary computing device.

FIG. 15 illustrates an example of derivation of IVC axes based on area and collapse parameters according to an embodiment disclosed herein.

FIG. 16 illustrates an example of estimation of venous return or cardiac output based on change in vessel areas according to a further embodiment disclosed herein.

FIG. 17 shows examples of signal traces used in determination of cardiac output according to embodiments disclosed herein, wherein trace (a) is a measured area trace, trace (b) is a filtered respiration component trace, and trace (c) is a filtered cardiac component trace.

FIG. 18 is a flow diagram illustrating another alternative embodiment for cardiac output estimation based on sensor data.

FIG. 19 shows further examples of signal traces used in determination of cardiac output according to embodiments disclosed herein, wherein trace (a) is a measured area trace, trace (b) is a simulated respiration component trace, and trace (c) is a simulated cardiac component trace.

FIGS. 20A-D illustrate an example of use of systemic vascular resistance based on sensor data and blood pressure data showing trends as a function of days for a heart failure patient experiencing symptoms according to an embodiment disclosed herein, wherein FIG. 20A shows SVR, FIG. 20B shows IVC area mean, FIG. 20C shows respiration rate, and FIG. 20D shows daily diuretic dosing.

### DETAILED DESCRIPTION

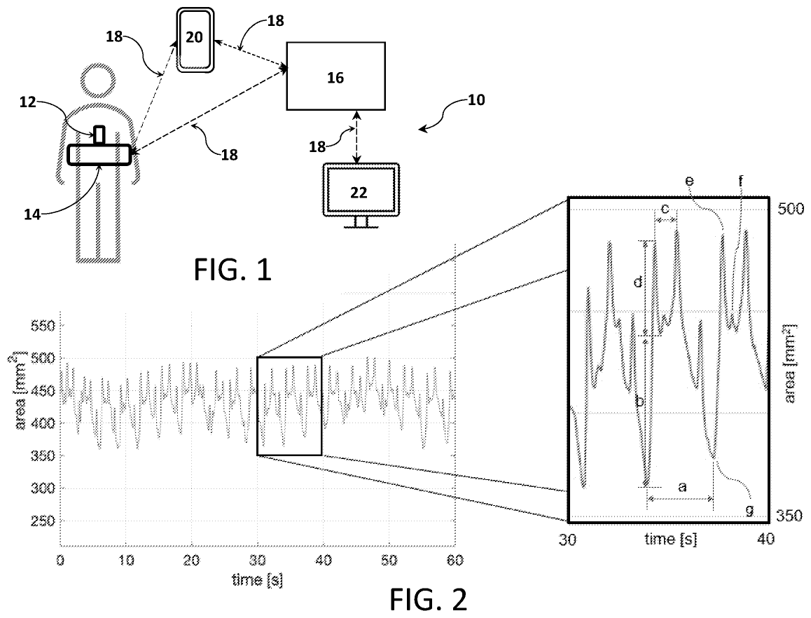

FIG. 1 depicts at a high level a system 10 for receiving and analyzing hemodynamic sensor traces to assist in providing more accurate diagnosis and improved treatment of cardiac-related conditions, particularly in heart failure patients. In general, systems in accordance with embodiments described herein will include a sensor subsystem that produces a signal representing patient hemodynamic function, a data analysis subsystem that receives signal data from the sensor subsystem, processes the received data to automatedly generate diagnostic and treatment recommendations, and an interface subsystem that permits patient and healthcare provider interaction with the system. System 10 as shown in FIG. 1 exemplifies one such embodiment, including an implanted sensor 12 and a patient-worn processing device 14 that receives the raw sensor signal and provides initial signal processing. Processing device 14 communicates with further processing platform(s) 16 via wireless communications links 18. Between processing device 14 and processing platform(s) 16, system 10 reads and interprets cardiac-health-state information contained within the sensor signal to provide diagnostic and treatment recommendations based on the extracted information. Data communication may be optionally facilitated through a patient personal device 20 such as a phone or tablet, which also may function as an input and output device for the patient as further described herein. An optional healthcare provider device 22 also may be provided to facilitate healthcare provider interaction with the patient and the system as further described herein.

Sensor 12 may comprise an external sensor system or an implanted sensor system. Examples of sensor 12 include vascular dimension sensors, such as an IVC area or diameter sensor, and vascular pressure sensors. With respect to vascular dimension sensors, a number of different sensor types may be used to produce an area trace signal including, for example, implanted variable inductance coils and implanted or external ultrasound devices. In one specific implementation, sensor 12 is an implanted wireless resonant circuit sensor and processing device 14 comprises a belt antenna as described, for example, in the incorporated U.S. Pat. No. 10,806,352. Other sensor types, such as implanted or external ultrasound, and implanted resistance-based sensors, may be employed as described in the foregoing incorporated patents and published applications.

Communication links 18 may be wired, wireless or a combination thereof based on the specific configuration of a system in accordance with the present disclosure. Persons skilled in the art may configure an appropriate data transmission protocol for communication link 18, selected from among many available standards. Communication links 18 are preferably bi-directional communication links. For example, a personal area network (PAN) connection such as Bluetooth may connect sensor processing device 14 to patient personal device 20. In some embodiments, personal device 20 may contain one or more software applications to perform signal processing functions with respect to the sensor signal. In other embodiments, personal device 20 may in this system merely act as an edge device to facilitate communication with processing platform (16) configured as cloud platforms, with communication occurring via cellular data links. Communications links 18 between different system platforms and components also may comprise internet connections to provide data transfer, for example between a cloud platform comprising processing platform(s) 16 and healthcare provider device 22. In some embodiments, all or part of the functions of processing device 14 also may be executed as a cloud-based computing device.

An example of an IVC area signal trace produced by system 10 is shown in FIG. 2. In general, as used herein area trace refers to a signal that presents vessel area as a function of time over a specific discrete time period corresponding to the trace reading and contains data representing cardiac and respiratory function, among other features. Processing platform(s) 16, such as diagnostics engine 102 described below, extracts that data by interpreting the features of the area trace. Some features of the area trace are identified in the enlarged box portion of FIG. 2. As shown therein, feature (a) represents the interval time per respiration cycle (t_{i,resp}). Feature (b) represents the area magnitude of respiration modulation. Feature (c) represents the interval time per cardiac cycle (t_{i,card}). Feature (d) represents the area magnitude of cardiac modulation in the IVC. Feature (e) represents the dominant cardiac peaks. Feature (f) represents the second cardiac peak of same cardiac cycle as the preceding peak (e). Feature (g) represents the respiration-related area reduction of the IVC.

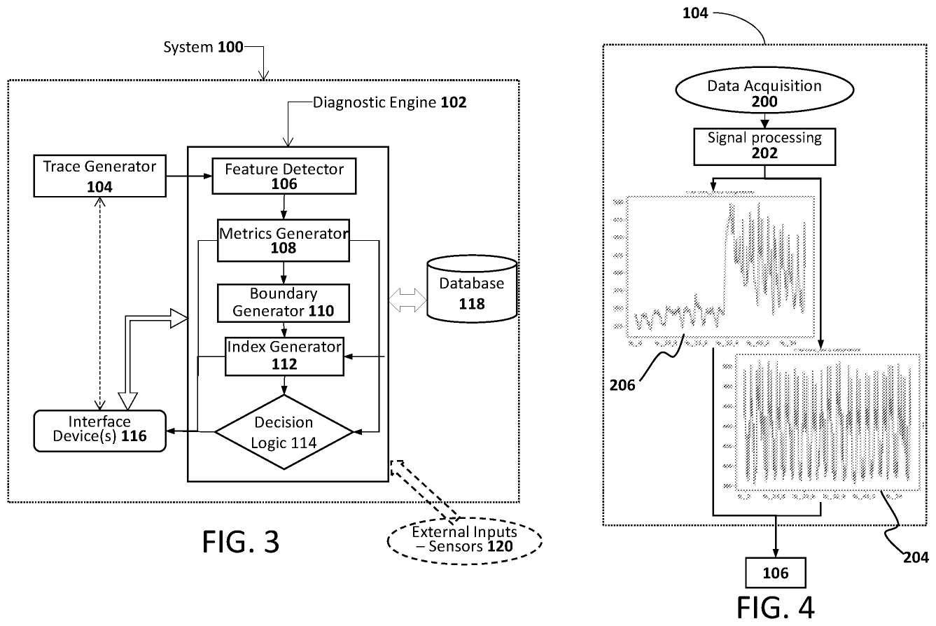

Persons of ordinary skill in the art may derive many different ways to configure and execute system 10 of FIG. 1 based on the teachings of the present disclosure. One example embodiment is exemplified in FIG. 3 as system 100. As illustrated therein, a sensor subsystem of system 100 comprises area trace generator 104, a data analysis subsystem comprises diagnostic engine 102, including modules such as feature detector 106, metrics generator 108, boundary generator 110, index generator 112 and decision logic 114, and an interface subsystem comprising one or more interface devices 116 to facilitate user interaction with the system, including uploading of patient specific data and receiving reports and notifications on patient cardiac health state. Diagnostic engine 102 also may communicate with one or more databases 118 and may receive additional patient-related information as external inputs from other patient sensors 120.

Trace generator 104 is a combined hardware and processing device comprised of a data acquisition device 200 and signal processing device 202 as depicted in FIG. 4. Data acquisition device 200 may comprise one or more of the aforementioned sensors 12. Signal processing device 202 is a signal amplifier/processor configured to receive raw data signals from data acquisition device 200 and to produce readable area trace signals suitable for communication and further processing within diagnostic engine 102. One example of a signal processing device is shown in FIG. 4 of the incorporated U.S. Pat. No. 10,806,352. In other examples, signal processing device 202 may comprise a software app executed on a patient personal device or a cloud platform. Examples of area traces produced in signal processing device 202 for communication to feature detector 106 include a regular respiration trace 204 and a maneuver trace 206.

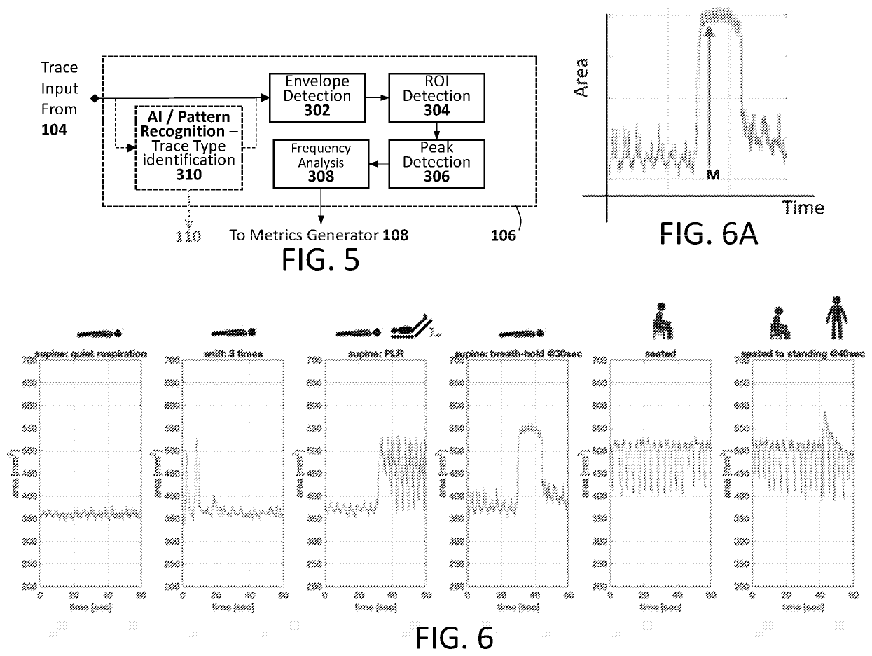

Feature detector 106 is a device such as circuitry, software or combination thereof that extracts relevant data from the area trace signal. Extracted data typically will include magnitudes and timing for selected features identified in an area trace through the reporting period (as shown as 60s trace in FIG. 2); that is, plural of interval time per respiration cycle, area magnitude of respiration modulation, interval time per cardiac cycle, area magnitude of cardiac modulation, dominant cardiac peaks, second cardiac peaks, and respiration-related area reduction. As depicted in FIG. 5, functional components for this purpose, which are generally understood and configurable by persons skilled in the art, may include an input signal integrity check, envelope detection 302, region of interest (ROI) detection 304, peak detection 306 and frequency analyzer 308. In some embodiments, feature detector 106 also may be an artificial intelligence-driven pattern recognizer 310. Pattern recognizer 310 employs machine learning and pattern analysis techniques to identify specific patterns in the area trace, which may be relevant in normalization and in diagnostic and treatment determinations as discussed more below.

In one embodiment, pattern recognizer 310 compares incoming area trace signals with known area trace patterns to determine whether the incoming area trace is reflective of a feature such as a signal response to a patient maneuver. “Maneuver” as used herein refers to a physical action taken by a patient, on his or her own initiative or in response to instructions, which stimulates an identifiable perturbation of IVC area. Some examples of area traces for different patient maneuvers are shown in FIG. 6, including supine: quiet respiration, sniff, supine: PLR (passive leg raise), supine: breath-hold, seated and seated to standing. An enlarged view of the area trace pattern for supine breath hold is shown in FIG. 6A. Area trace patterns representing different maneuvers may be stored, such as in database 118 (FIG. 3), and accessed by pattern recognizer 310. In one embodiment, this was achieved by transforming trace signals into a bag-of-words and applying a Bag-of-SFA-Symbols (BOSS) model. The model was then trained to detect maneuver-relevant features from such bag-of-words in order to classify maneuver/no maneuver with an accuracy of better than 80%. Knowledge of the type of trace pattern may be utilized by boundary generator 110 as further described below. In another embodiment pattern recognizer 310 can be used to identify features within the area trace signals that have been shown to be predictive of clinical occurrences such as atrial fibrillation or tricuspid regurgitation for example.

Feature detector 106 also may perform data integrity checks. Features of the area trace signal may be used to confirm if the system has been used correctly. For example, quality checks could be trained on supervised data as, for instance, the type of maneuver prescribed. Models predicting maneuver type or artifact can then be used to quality check every single area trace to avoid deriving corrupted information such as excess movement during recording, insufficient quantity or quality of maneuver performed, etc. Auxiliary sensors such as accelerometers, blood pressure, weight, activity monitors, patient input can also be used to facilitate data integrity checks.

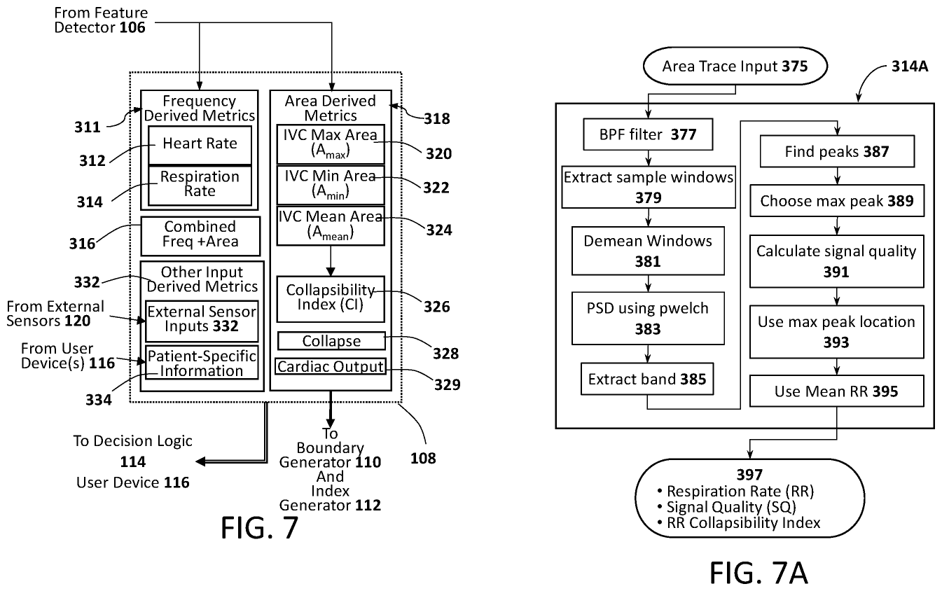

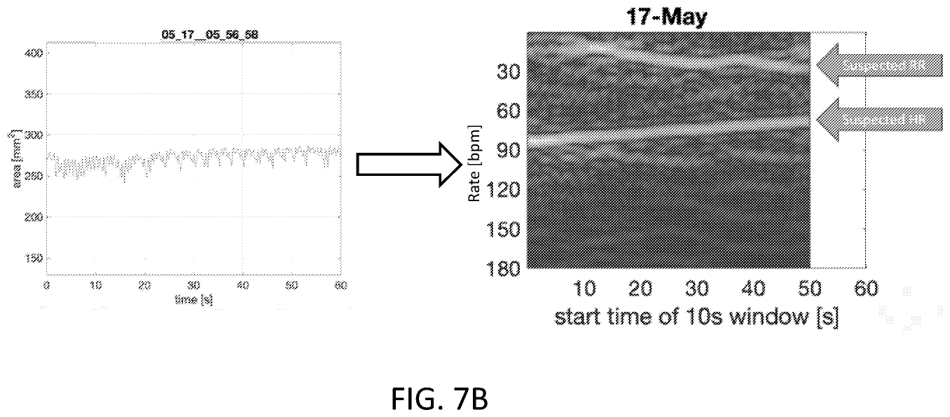

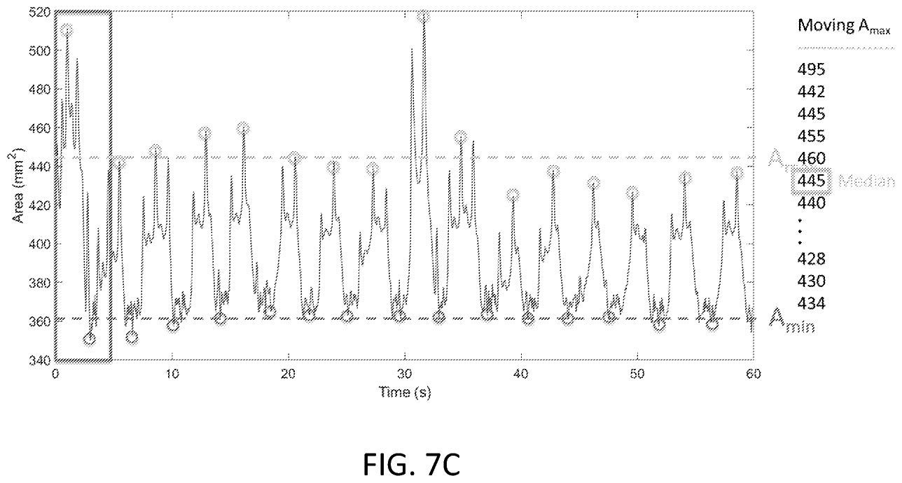

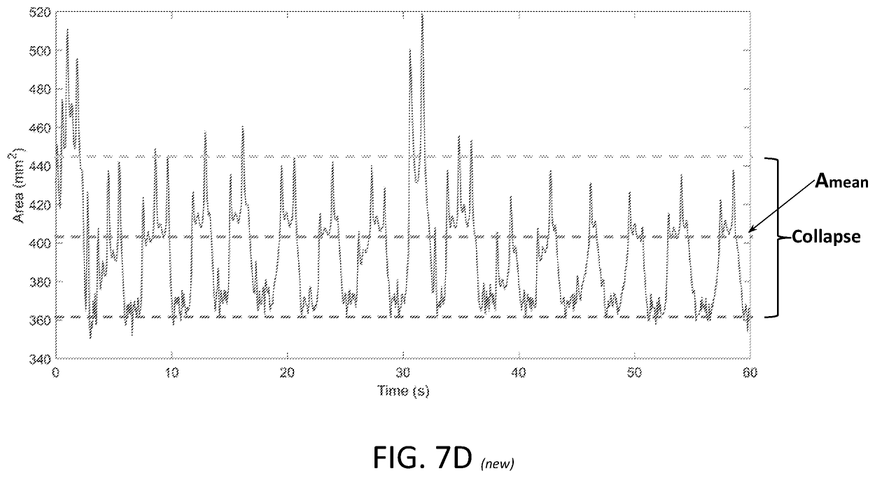

Data extracted from the area trace signal by feature detector 106 is provided to metrics generator 108. Metrics generator 108 is a software-based machine configured with a number of different modules, as shown in FIG. 7, that generate specific cardiac-related metrics on which cardiac-related diagnostic and treatment determinations can be based. Included within frequency-derived metrics group 311 are at least heart rate module 312 and respiration rate module 314. Based on information received from feature detector 106, heart rate module 312 determines heart rate (HR) based on the relationship:

$$H R = 1 / ( {t}_{i , card} ) \qquad [ 1 ]$$

where t_{i,card} is the cardiac cycle interval time, which may use an average or median, feature (c) in FIG. 2.
Also based on information from feature detector 106, respiration rate module 314 calculates respiration rate (RR) based on the following relationship:

$$RR = 1 / ( {t}_{i , resp} ) \qquad [ 2 ]$$

where t_{i,resp} is the respiration cycle interval time, which may use an average or median, feature (a) in FIG. 2.
An alternative embodiment for determination of respiration rate by extraction from the area trace is shown in FIG. 7A and discussed in more detail below.

Included within area derived metrics group 318 are at least three area determination modules, and collapsibility index module 326. Additional optional modules may include modules for determination of collapse 328 and cardiac output 329. Maximum IVC area (Aₘₐₓ) module 320 may determine Aₘₐₓ based on the value of the largest dominant cardiac peak, feature (e) in FIG. 2, occurring during the relevant sampling period. Minimum IVC area (Aₘᵢₙ) module 322 may determine Aₘᵢₙ based on the value of the greatest respiration area reduction valley, feature (g) in FIG. 2, occurring during the relevant sampling period. Alternatively, the maximum and minimum area values are extracted from a trace by finding the global maximum and minimum, excluding trace sections that are unusual in the light of the overall trace/excluding area sections of the trace that may have been corrupted by artifacts. An example of this technique is illustrated in FIG. 7C. Mean IVC area (Aₘₑₐₙ) module 324 determines Aₘₑₐₙ based on the maximum and minimum areas (one half the sum of Aₘₐₓ and Aₘᵢₙ), as shown, for example, in FIG. 7D.

Collapsibility index (CI) module 326 of metrics generator 108 uses the determined area parameters to determine collapsibility index for the IVC based on the relationship:

$$CI = ( {A}_{max} - {A}_{min} ) / {A}_{max} * 100 \% . \qquad [ 3 ]$$

In a further alternative embodiment, collapse (identified on the area trace in FIG. 7D) is separately included via collapse module 328, wherein collapse (Collapse) is determined based on the following relationship:

$$Collapse = {A}_{max} - {A}_{min} \qquad [ 4 ]$$

Thus, collapsibility index also may be stated as:

$$CI = Collapse / {A}_{max} * 100 \% \qquad [ 5 ]$$

For example, respiratory collapse can be determined from the area trace. This has the potential advantage that it is another somewhat independent signal that can be used to predict volume or congestion status or pressure—low collapse at high volume/pressure, high collapse at euvolemia/normal pressure, and low collapse at hypovolemia/low pressure giving an ‘n’ shaped collapse vs. area curve. Trending of features of the raw trace or the maneuver traces may also prove useful, i.e. accelerating increase in area may necessitate more urgent or severe action than gradual increases. In another example, other frequency-based signals can also be extracted and could be used as inputs to the calculation. As a further example, it is known that the IVC area signal changes with each breath of the patient and therefore the low frequency oscillation of the IVC can be used to extract the respiration rate. Respiration rate has been shown to be predictive of heart failure status and would therefore be a strong input into the overall patient status estimation. Additionally, metrics combining frequency and area inputs 316 may be determined.

Inputs from wearable devices such as activity trackers can also be predictive of cardiac patient outcomes and can also be integrated into the calculation with reduced activity being a predictor of worsening status. Also, weight is a factor that may be used in prediction of heart failure decompensation, for example a gain in the region of more than 2 kg in 2 days may be considered significant. Patients taking their daily weight can also be integrated into the calculation. Sleep, activity, heart rate variability, blood pressure and other wearable outputs could also be integrated into the Congestion Index. Additionally, data from the implanted sensor and its overall system 100 can be integrated into wearable devices' systems as a source of additional data for the predicition and/or monitoring of health status.

Included within the other input-derived metrics group 332 are metrics derived from other external sensors (meaning external with respect to system 100, which may include both in vivo and ex vivo sensors), such as pulse oximetry, temperature, blood pressure, urine output, cardiac output, and catheter pressures, etc. Patient-specific information 334 generally comprises information about patient physiological parameters such as height, age, weight, sex and may also comprise current activity information, input through a user interface by a patient or care provider. Another alternative external sensor input is accelerometer readings from a patient-worn component of the sensing system, such as patient-worn processing device 14, which may be embodied as an antenna belt as described in incorporated patent publications. Such accelerometer readings can be used to determine patient position and activity/motion during a trace period and thus increase accuracy of metrics derived from the trace.

As shown in FIGS. 3 and 11, metrics from metrics generator 108 also may be directly provided to decision logic 114 for application per specific diagnostic or treatment algorithms. Metrics also may be provided to interface device 116 for display and monitoring by user. Area-based metrics are also provided to boundary generator 110 for use in boundary determination.

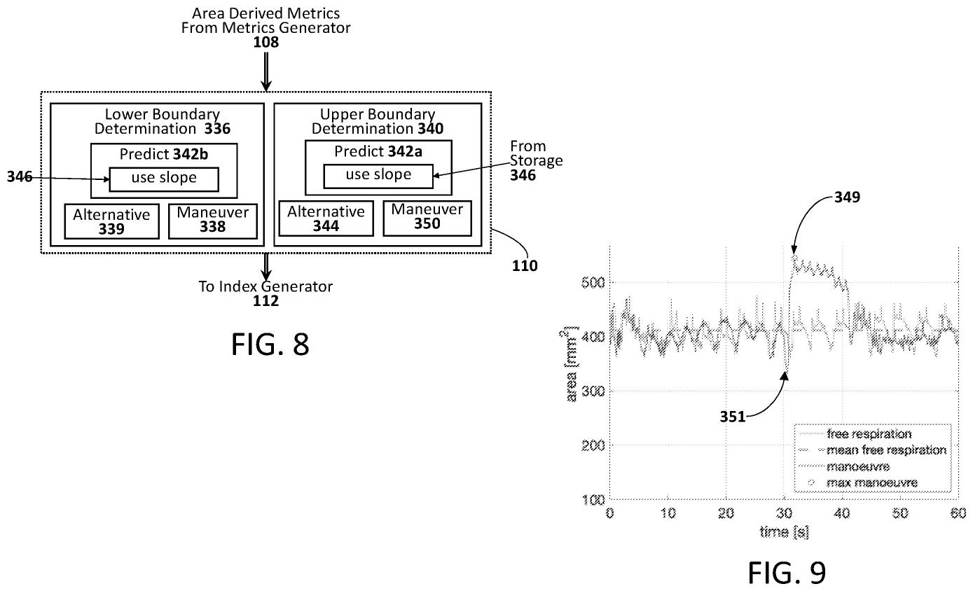

In order to overcome the calibration challenges created by intra-individual and inter-individual variations arising from heterogeneity of vessel shape as mentioned above, embodiments of the present disclosure generate specific reference points against which periodic IVC area readings can be compared to assess current patient fluid state and related cardiac health. In one example, boundary generator 110, shown in FIG. 8, uses IVC area data extracted or predicted from the area trace to determine upper and lower boundaries for this purpose. Boundaries may be considered as static or dynamic. Static boundaries are maximum/absolute values that would not be expected to significantly change over longer time periods. The absolute max/min area values represent upper and lower edges of the individual IVC area range can be considered as static boundaries. Absolute maximum area may be related to total circulating blood volume. It could be assumed that these boundaries are anatomical constants, that the IVC in a specific patient can always only get to be a certain size. In this way the boundaries can be considered to be static over time. This allows a single maneuver to be used to understand a patient's IVC range and those boundaries be used over time. This essentially means that a single calibration of the feature is performed. Static boundaries may be used, for example, in relationship to a specific set of conditions to be diagnosed.

On the other hand, dynamic boundaries may change over shorter time periods. Dynamic boundaries do not necessarily indicate absolute anatomical limits, but represent the current limits arising out of changing physiological parameters such as venous tone, and/or intra-abdominal pressure. Dynamic boundaries thus may represent new and clinically relevant limits for each quiet respiration reading or occasionally when estimation is available from maneuver or other means to use the information that is in closest proximity time-wise to the readings used for volume status assessment. In other words, based on overall patient fluid state in terms of total fluid distribution between vascular and extravascular fluid, dynamic boundaries as defined herein may represent a more clinically relevant basis for assessing patient fluid state at the time of a specific reading. Furthermore, dynamic maximum area boundary may be related to right atrial pressure or vascular tone.

As shown in FIG. 8, boundary generator 110 includes two components for generation of boundaries, lower boundary determination module 336 and upper boundary determination module 340. In various embodiments, lower boundary determination 336 and upper boundary determination module 340 may be alternatively configured to determine the lower and upper boundary based on a maneuver or to predict the boundary based on regular respiration. In a further alternative, lower and upper boundary determination modules 336 and 340 may comprise both maneuver determination sub-module 338, 350 and prediction sub-module 342a,b. FIG. 9 illustrates the operation of maneuver determination sub-modules 338, 350, showing an area trace for an inspiration breath-hold maneuver overlaid over a regular respiration area trace. Lower boundary determination 336 accounts for any physiological or device-related restriction preventing the IVC from full collapse, e.g. sensor radial force, sensor positioning across a venous branch, or other physically restricting features of the anatomy or implanted device. Sub-module 350 may receive information on the type of maneuver in a number of ways. For embodiments employing a feature detector 106 including trace pattern recognizer 310, the system may provide the maneuver type information directly. Alternatively, a user such as a care provider, when instructing the patient to perform the maneuver, would also input information via an interface device 116 informing of the instruction to perform a maneuver. Trace pattern recognizer 310, where present, may also be used to confirm patient compliance with the maneuver instruction by comparing the received area trace pattern with stored patterns for the instructed maneuver. Regardless of the source, when informed of the performance of a maneuver, maneuver determination sub-module 350 identifies the maximum area in the maneuver area trace, point 349 in FIG. 9, as the upper boundary.

In some situations it may not be practical or desirable to require the patient to perform a maneuver. Prediction sub-module 342a is configured to predict the upper boundary (UB) based on a normal respiration area trace based on the following relationship developed by the Applicant:

$$UB = ( - 1 / slope ) \cdot CI + {A}_{mean} \qquad [ 6 ]$$

In one alternative, the “slope” used in Eq. [6] is a constant reference slope of −0.18%/mm² as identified in Huguet et al., Three-Dimensional Inferior Vena Cava for Assessing Central Venous Pressure in Patients with Cardiogenic Shock, JAm Soc Echocardiogr. 2018; 31: 1034-43 (https://doi.org/10.1016/j.echo.2018.04.003), which is incorporated by reference herein in its entirety. The slope used may be retrieved from data storage 346. In other alternative embodiments this slope could be individualized based on each patient's area and collapse data or may be trained on a collection of area and collapse or other data over some period of time. Further alternative techniques 344 for upper boundary generation also may be derived based on the teachings of the present disclosure.

Lower boundary (LB) determination module 336 in some embodiments also employs a maneuver determination sub-module 338 to identify the lower boundary as the minimum area point (351 in FIG. 9) in a maneuver area trace in substantially the same manner that upper boundary determination sub-module 350 identifies the upper boundary. For example, lower boundary determination may be made via sniff maneuver, or any other maneuver that reduces the IVC size, using methodology described above. Alternatively lower boundary prediction 342b uses individual patient or population area and collapse data to predict lower boundary using a method similar to upper boundary prediction 342a using Eq. [7]:

$$LB = ( - 1 / slope ) \cdot C + {A}_{mean} \qquad [ 7 ]$$

Further alternative sub-modules 339 may also be provided to derive the lower boundary value from a population basis as the average minimum sensor size measured in the IVC, or sensor information regarding IVC area at given equivalent pressures, such as by comparing sensor derived area/pressure curves with standard area/pressure curves (determined from radial or flat plate force information) and be a programed constant for all patients.

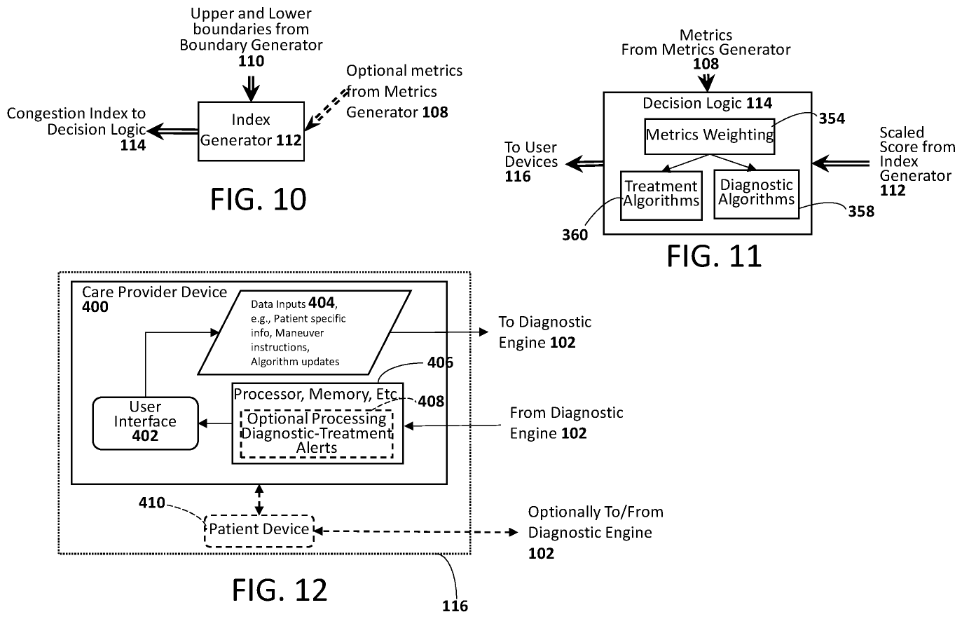

Upper and lower boundaries generated by boundary generator 110 are provided to index generator 112, shown in FIG. 10. These boundary values enable normalizing the volume measurement of the vessel to return a number ranging from 0% to 100% or 0 to 10, with 0% being close to 0 mmHg internal filling pressure or low volume/low congestion/hypovolemia and 100% being close to the maximal possible filling pressure in this vessel (˜20 mmHg) or high volume/high congestion/hypervolemia. Index generator 112 assigns the boundaries to a relative scale with the lower boundary set as the scale minimum and the upper boundary set as the scale maximum. In some embodiments, the relative scale is established as a 0-100% scale. Once the relative scale is established, current area readings are scored on the relative scale, for example, an area reading falling half way between the upper and lower boundaries would be scored as 50%. Because each patient is capable of displaying a range of IVC areas based on a number of factors including, but not limited to, anatomical size, current fluid status, body position, vascular tone status, and cardiac and abdominal pressures, normalization as described herein accounts for these parameters to produce a more standardized Congestion Index that is comparable across time for the patient and comparable across patients to help facilitate more accurate diagnosis and treatment of heart-failure-related conditions. As described above, at the outset, the new IVC area-based metrics disclosed herein may not be well-understood and correlations to well-known physiological measures can aid in the clinical understanding of the early adopters and ultimately the clinical masses. In one embodiment, the Congestion Index may be determined based on the relationship of mean area and the upper and lower boundaries as in equation [8]:

$$Congestion Index = 100 * ( ( {A}_{mean} - LB ) / ( UB - LB ) ) \qquad [ 8 ]$$

In another example embodiment, index generator 112 uses the Lower Boundary determined from Boundary Generator 110 with Collapsibility Index 326 to produce a vascular Congestion Index using the following equation:

$$Congestion Index = ( {A}_{max} - {A}_{min} ) / ( {A}_{max} - LB ) * 100 \% \qquad [ 9 ]$$

An alternative of this equation might be to subtract from 100 in order to reverse the direction of the index resulting in the equation:

$$Congestion Index = 100 - ( {A}_{max} - {A}_{min} ) / ( {A}_{max} - LB ) * 100 \% \qquad [ 10 ]$$

In yet another alternative, Congestion Index may be determined using the upper boundary alone as the ratio of mean area to the upper boundary as follows:

$$Congestion Index = 100 * ( {A}_{mean} / UB ) \qquad [ 11 ]$$

In a further alternative embodiment, index generator 112 optionally receives and factors metrics from metrics generator 108 and boundary generator 110 into the normalized IVC Congestion Index. In such an embodiment, index generator 112 may compress multiple features into a single metric. For example, index generator 112 may be configured to output a single number within a standardized range for each patient. This single number output may be an indicator of a measurable variable such as RAP, worsening heart failure, impending hospitalization, probability of an impending event, a prompt to change medication, etc. This output will be common to all patients and contain or summarize the information from all of the inputs to provide an actionable output for clinicians. In a way this is comparable to scales such as temperature with 37 degrees Celsius as an accepted normal level and accepted high and low thresholds. In such a configuration, index generator 112 may output a Congestion Index ranging from 0 to 100% indicating the risk of an impending heart failure hospitalization within a selected time period, for example, the next thirty days.

The IVC Congestion Index produced by index generator 112 can be directly reported by user interface devices 116, but also can be a valuable input for decision logic 114 as shown in FIG. 11. Decision logic 114 comprises one or more instruction sets for determining patient heart health status and treatment recommendations based on heart function parameters generated by metrics generator 108 and the Congestion Index. Decision logic 114 may be embodied in one or more circuits, executed by one or more data processing devices or distributed across a combination of such platforms. In general, the Congestion Index, optionally along with other metrics from metrics generator 108 are selectable inputs to various diagnostic and treatment algorithms 358, 360 executed in decision logic 114. In one example, the thresholds may be set as follows:
0 to 30%: Below normal range which could indicate least likely risk of heart failure hospitalization due to volume overload, yet risk of hypovolemia, patient data needs attention and review, potentially reduce diuresis
30% to 70%: Normal range, potentially optimize guideline directed medical therapy
70% to 100%: Above normal range which could indicate elevated risk of impending heart failure hospitalization, patient data needs attention and review, potentially increase diuresis
   In this manner, any one feature of daily Congestion Index with selected cut-off thresholds (i.e. lower and upper) is now scaled onto the final Congestion Index as follows:
(1) The lower range from 0 to lower threshold will be scaled from 0 to 30%
(2) The normal range from lower threshold to upper threshold will be scaled from 30 to 70%
(3) The high range from upper threshold to 100% will be scaled from 70 to 100%

Utilizing an approach as outlined above, as an example, a priority list algorithm can be created for moving patients into a higher priority status after a threshold crossing for a defined number of days and removing patients when within normal range for a defined number of consecutive days. Also, the primarily area-based Congestion Index as described above can be enhanced by factoring additional heart function-related parameters as further described below to provide a potentially fuller picture of patient heart health status. Other examples of such algorithms are disclosed in the aforementioned and incorporated U.S. Pat. No. 11,564,596 and alternative embodiments could inform the up or down titration for specific medications, such as diuretics, vaso-dilators or other medications, based on a pre-specified, patient specific prescription. Additional examples of conditions that may be diagnosed and treatment recommendations generated via algorithms 358 and 360 include conditions such as tricuspid regurgitation, atrial/ventricular tachycardia/bradycardia.

In other embodiments, maneuvers or similar changes can be utilized to move the patient between two or more different states. Signal differences between these states can be indicators of different physiological conditions. One example of this would be in the case of HFpEF patients where deterioration of condition parameters is only evident with exercise. In this case the no exercise and exercise states could be used to provide a signal to diagnose the presence of HFpEF. Bendopnea is another example where a maneuver can result in shortness of breath and this could also be evidenced via the signal indicating respiration rate/magnitude changes. These maneuvers could be detected from the signal in feature detector 106 or could be inputs from the interface device 116.

Decision logic 114 may utilize any number of metrics as inputs. In some cases only individual metrics or groups of related metrics, such as area metrics, may be used. In one alternative embodiment, metrics weighting sub-module 354 applies a weighting factor to one or more of the individual metrics inputs. Weighting factors may be determined via analysis of clinical and pre-clinical data and pre-specified or could be continually calculated and updated based on user-specific input. Notifications in this case may comprise instructions to take treatment actions, instructions to take additional readings or instructions to a care provider to alert the care provider to possible clinically problematic situations.

As illustrated in FIG. 12, it is contemplated that interface devices 116 will comprise at least a user interface 402 accessible to a care provider, for example via care provider device 400, for data inputs 404 such as patient-specific information, prescription details, user settings, maneuver instructions, algorithm updates and other system hygiene, etc. User interface 402 could also provide notifications generated by decision logic 114 or other components of diagnostic engine 102 and could be web-based or a mobile application. In some embodiments, interface devices 116 may include processing capabilities in the form of one or more processors 406 and associated hardware/software. Diagnostic or treatment alerts 408 also may be delivered through care provider device 400, for example as described in the aforementioned and incorporated U.S. Pat. No. 11,564,596. Interface devices 116 also may encompass patient personal devices 410, which may be employed for a variety of functions in the system in addition to patient notifications, as shown, for example, in FIG. 1.

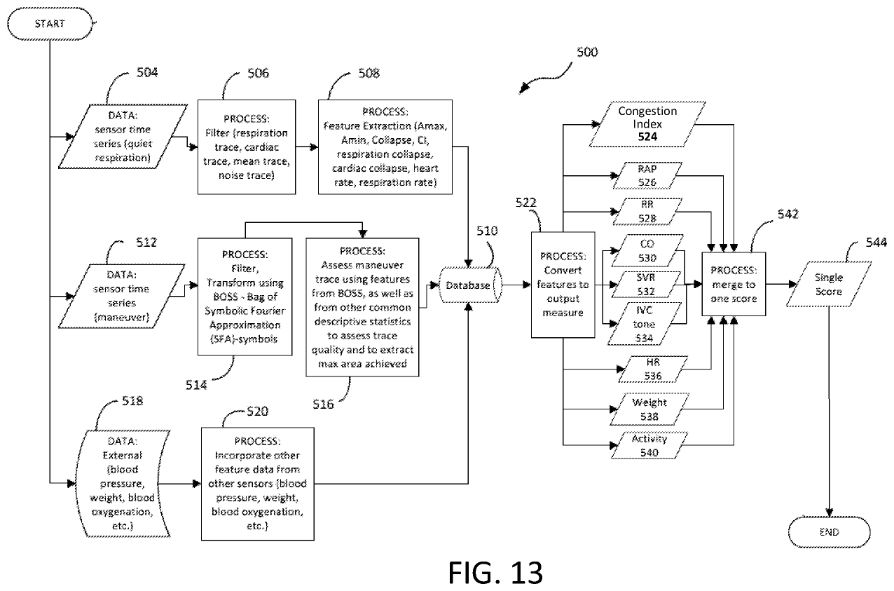

Embodiments of the present disclosure also may be described in terms of a continuous process flow, such as example process flow 500 shown in FIG. 13, or alternative embodiments of the system may have versions of this formation. As shown therein, different data types invoke different processes. For example, data 504 representing a sensor time series for quiet respiration invokes process 506 to filter (respiration trace, cardiac trace, mean trace, noise trace) and process 508 for feature extraction (Amax, Amin, Collapse, CI, respiration collapse, cardiac collapse, heart rate, respiration rate). Similarly, data 512 representing a sensor time series for a maneuver invokes process 514 to filter and transform using BOSS (Bag of Symbolic Fourier Approximation (SFA)-symbols) and process 516 to assess the maneuver trace using features from BOSS, as well as from other common descriptive statistics to assess trace quality and to extract max area achieved. External data 518, such as blood pressure, weight, blood oxygenation, etc., invokes process 520 to incorporate such data representing other externally sensed features from other sensors or direct inputs. Outputs of these processes may be stored in database 510.

Feature outputs from database 510 (or directly from upstream processes), invoke process 522 to generate metrics as described herein, including, for example, Congestion Index 524, Right Atrial Pressure (RAP) 526, Respiration Rate (RR) 528, Cardiac Output (CO) 530, Systemic Vascular Resistance (SVR) 532, IVC tone 534, Heart Rate (HR) 536, Patient Weight 538, Patient Activity 540, and other metrics described herein. Metrics as generated are then processed 542 to produce an all metrics-based or enhanced IVC congestion index score 544, which is presented to a user or used as an input to decision logic 114 as a basis for further diagnostic or treatment decisions.

In some embodiments, the enhanced indexing process 542 is configured as an AI or machine-learning-based model that is trained based on all described inputs and determines the best weightings for each metric in order to predict a specific output such as “worsening heart failure”, “increasing congestion”, likelihood of hospitalization”, etc. One such example would be a multiple logistic regression model that uses a training dataset to develop a model which returns the probability of hospitalization based on the inputs or a subset of the inputs 524-540. Another embodiment would be a decision tree model that splits the data from each of the inputs based on the training set data to categorize into “high likelihood of hospitalization” or “no likelihood of hospitalization” for example, repeating this process for each input parameter until the model is created to accurately predict likelihood of hospitalization.

Further alternative embodiments for generation of various metrics within components of metrics generator 108 are described below with reference to FIGS. 7A-B and 15-20A-D. For example, an additional alterative embodiment for respiration rate module 314 of metrics generator 108 provides for extraction of respiration rate from an area trace as shown in FIG. 7A. As illustrated therein, respiration rate module 314A receives an area trace as input 375. Baseline wander and higher frequency components are then removed from the input signal using a butterworth bandpass filter module 377. An example range for this filter would be 0.08 Hz to 0.75 Hz. The signal is then split into many windows with a certain lag module 379 an example would be five hundred twelve (512) windows with a four (4) second lag. The mean is then subtracted from each window module 381. These windows from 381 are then used to estimate the power spectral density. This is done using Welch's method to account for possible non-stationarity in the signal, i.e. a change in respiration rate module 383. For example, this could use a Hann window with a sample size of two hundred fifty-six (256). The power spectral density from module 383 is then filtered to examine only possible respiration rates module 385 for example eight (8) to forty (40) beats per minute. All peaks in this filtered spectra are then found in module 387 for example using peak prominence. The largest detected peak is extracted module 389. The signal quality of this peak is then assessed by comparing it with signal quality outside of the range excluding potential harmonics of this peak. If the signal quality is greater than a threshold in module 391, the breaths per minute as estimated by the max peak is accepted for the window else the respiration rate for the window is rejected at module 393. Module 383, 385, 387, 389, 391 and 393 are repeated across all windows given by 381. Module 395 takes all of these respiration rates in breaths per minute as an input. If the number of respiration rates is greater than a threshold, the mean of the accepted respiration rates is returned as the global respiration rate for the input 375. A final output 397 of this respiration rate module 314A returns an individual value for each of respiration rate (RR), signal quality (SQ) and respiration collapsibility index, derived for each area trace input.

As yet another metrics example, each beat of the heart also perturbs the IVC and this higher frequency signal can also be extracted from the IVC area trace by spectrally decomposing the area trace using Fast Fourier Transformation (FFT) as shown in FIG. 7B. It is well known that heart rate is also a useful metric in the management of patients and this could be included in the generated metrics. Increased or decreased rate or increased rate variability can be predictors of worsening outcomes. Also, pacing related metrics may be determined from the area trace.

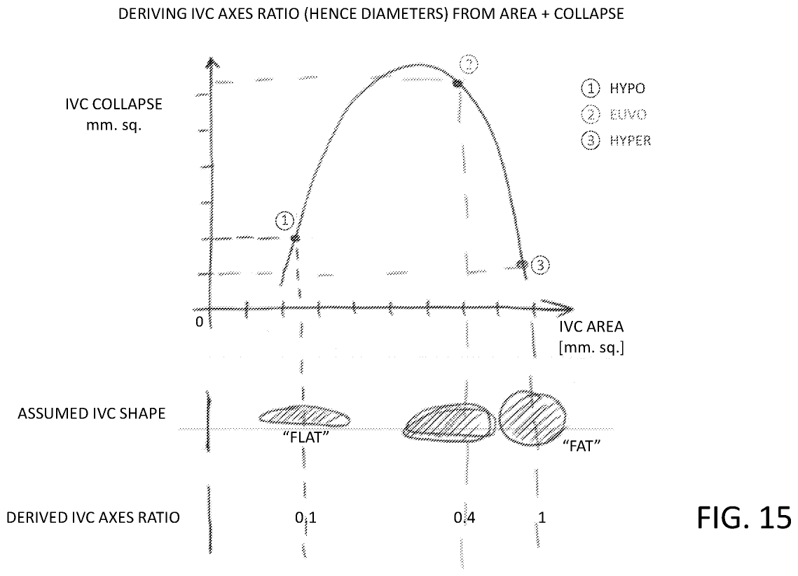

In another example, as illustrated in FIG. 15, assuming an idealized ‘n’ shape curve for IVC area vs. IVC collapse, it may be possible to estimate the ovality of the IVC (hence provide an approximation of the IVC diameter should the existing diameter-based clinical guidelines be used for thresholds). Also, it may be assumed that the IVC shape varies from flattened ellipse to fully circular along that ‘n’ curve, with axis ratio correspondingly varying from low (˜0.1) to equal (1) along the curve. Thus, knowledge of both collapse and area allow approximate determination of location on ‘n’ shape, which in turn allows estimation of the IVC's axis ratio. The approximated axis ratio can in turn be plugged into the area equation for an ellipse, reducing the number of unknown variables to 1 thereby permitting an exact solution for IVC major and minor diameters (for comparison with existing clinical guidelines).

In another embodiment, the area change seen in the IVC is used to estimate venous return (VR)/cardiac output (CO) by measuring the change of area per time as the integral under the curve (AUC) and a simple initial calibration to a clinical cardiac output measurement. This measurement is based on volume passing through the IVC causing temporal distension of the IVC relative to its minimum size. FIG. 16 illustrates this distortion in an idealized cross-sectional depiction of the IVC with (b) and (d), which corresponds to the same features in the area trace (FIG. 2), identifying circle and area shifts. In this example, cardiac output can be determined using the following equation [12]:

$$CO = VR = corfacresp ( Rcol * RR ) + corfaccard ( Hcol * HR ) \qquad [ 12 ]$$

Correction factors (corfacresp and corfaccard) are derived from calibration step reference cardiac output as measured (e.g. ultrasound to estimate cardiac output). The correction factors can be split into elements contributing to respiration and to cardiac as the distance of the device to the heart can affect the cardiac component detected relative to the respiration modulation. For example, two-thirds of bloodflow into the right atrium originates from the IVC. As blood flows through the IVC it expands being a compliant vasculature. Similarly to stroke volume estimates from images of the left atrium, the change in the IVC cross-sectional area is correlated with cardiac output as has been reported in 2004 by Barberi et al. [Intensive Care Med (2004) 30:1740-1746, DOI 10.1007/s00134-004-2259-8](incorporated herein by reference in its entirety). The IVC modulates with respiratory effort and cardiac activity. The concept linking IVC area change to cardiac output relies on four main features—area change with respiration and cardiac activity as well as with heart rate and respiration rate. Due to the complex nature of venous return/cardiac output—it is a time integral—it is necessary to calibrate the area time integral measured in m² in a way that it relates to cardiac output measured in litres/min. To do that at least one synchronised cardiac output measurement is required in conjunction with a recording of free respiration IVC sensor area. Assuming 0 m² and 0 litre/min is the second calibration point, a simple linear calibration is possible, ultimately leading to a conversion factor f in units of litres/min/m² so that:

$$CO = fcorr * AreaTimeIntegral ( Sensor ) \qquad [ 13 ]$$

In a different embodiment, multiple calibration points are created through phlebotomy/withdrawal of blood and re-infusion or through saline injection of a known bolus volume. The cardiac output accuracy is expected to improve when respiration modulation of area is related in a weighted manner to cardiac modulation of area. Furthermore, tricuspid regurge information can be incorporated to correct for regurge-related area modulations. Correction factor weighting may be included to adjust contribution from cardiac and respiration with seen collapse. This approach requires more than one single reference point for calibration. Cardiac output measurements during provocative maneuver such as, for instance, an inspiration or expiration breath-hold may allow separating the respiratory component from its cardiac component—as during breathhold the cardiac component is exclusively visible.

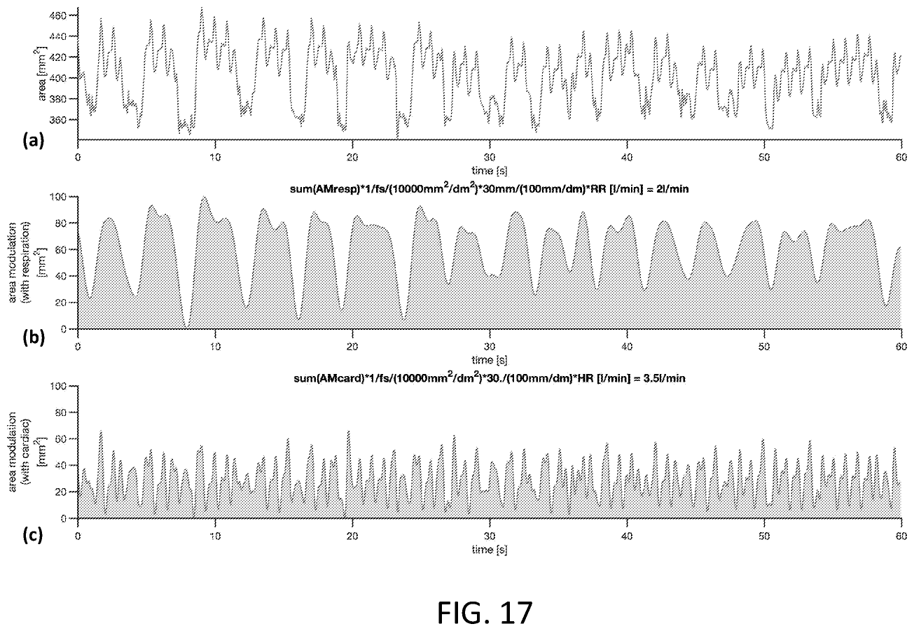

As further shown in FIG. 17, measured area trace and filtered respiration and cardiac component traces can be used to compute the area under the curve (AUC) as a metric for cardiac output. The change of area in the vessel is correlated to the volume delivered to the right atrium—that is generally known as venous return. Venous return is accepted to equal cardiac output. Venous return is composed of flow from superior and inferior vena cavae. As this sensor is intended in one of these locations, an initial calibration step is required. Common clinical measurement methods, such as ultrasound or thermodilution, and others can provide a reference cardiac output level. In some embodiments, a single cardiac output measurement could be used, or alternatively, a second cardiac output and sensor reading could be obtained via the use of a maneuver or other method. With such a reference and the calculated AUC, the correction coefficient to link both is established. The correction coefficient can be further refined by adding information about the respiration and cardiac contributions to flow in the respective sensor location, for instance, by splitting the single correction factor by a pre-determined ratio applied to cardiac and respiration.

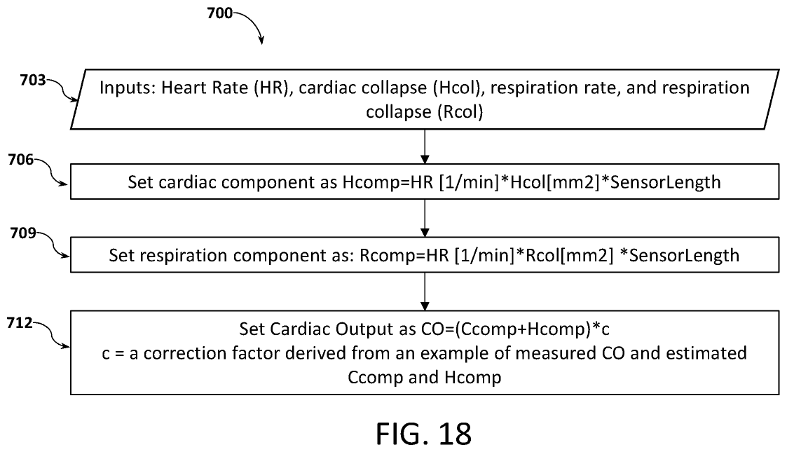

In another embodiment, as shown in FIG. 18, a cardiac output estimation process 700 may be used to estimate cardiac output directly from sensor data. Inputs 703 to process 700 include extracted features related to the area under the curve such as cardiac collapse (Hcol) and respiration collapse (Rcol), as well as heart (HR) and respiration (RR) rate. With these inputs, a cardiac component of output is set at 706 as the product of heart rate, cardiac collapse and sensor length. Similarly, the respiration component of output is set at 709 as the product of heart rate, respiration collapse and sensor length. Sensor length in this process may refer to the longitudinal length of the sensor coil portion of a wireless resonant circuit sensor implant that is configured and dimensioned to be implanted in a patient blood vessel in contact with the vessel wall. One example of such a sensor implant comprises an expandable and collapsible variable inductance coil with a plurality of adjacent wire strands formed around an open center to allow substantially unimpeded blood flow through the open center. In some such embodiments, the coil is configured and dimensioned (i) to extend around an inner periphery of the vessel when implanted therein and (ii) to move with the vessel wall in response to expansion and collapse of the vessel. The sensor implant also comprises a capacitance, which together with the variable inductance coil, forms a variable inductance resonant circuit having a variable characteristic frequency correlated to the diameter or area of the expandable and collapsible variable inductance coil. In further alternatives, the expandable and collapsible variable inductance coil comprises plural wire strands, and the inductance changes based on changes in cross-sectional area or diameter across the coil open center in response to expansion and collapse of said coil. Such sensor implants are described in more detail, for example, in incorporated-by-reference U.S. Pat. No. 10,806,352 (for example, RC-WVM implant 12 and height (A) as described therein corresponding to sensor length). It is to be noted that steps 706 and 709 may occur in any order or simultaneously. Total cardiac output is then set at 712 as the sum of the two component outputs multiplied by a correction factor. Persons of ordinary skill in the art may derive the correction factor based on the teachings of the present application using an example of measured cardiac output and correlated respiration and cardiac components.

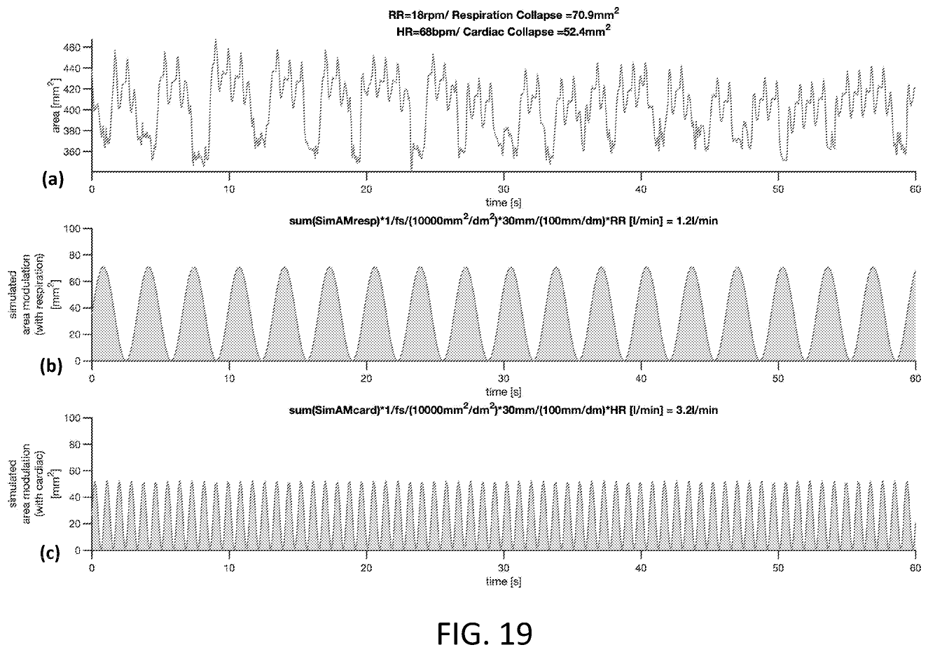

As further illustrated in FIG. 19(a)-(c), the parameters cardiac collapse (Hcol), respiration collapse (Rcol), heart rate (HR) and respiration rate (RR) can be used to calculate the respiration and cardiac traces synthetically (simulated area modulation) to again use the AUC method as another alternative means for assessing cardiac output. In this embodiment, the collapse component is used to inform the magnitude of the oscillation and the rate informs the oscillatory frequency. Thus the respiration trace (b) in FIG. 19 can be simulated as:

$$RespirationTrace = Rcol * sin ( 2 * pi * RR * TimeSamplesVector ) \qquad [ 14 ]$$

where the time samples are a linear range of values starting from a starting value to a final time stamp in discrete steps defined by the sampling frequency. In analogue, the cardiac trace (c) can be simulated as:

$$CardiacTrace = Hcol * sin ( 2 * pi * HR * TimeSamplesVector ) \qquad [ 15 ]$$

In one additional alterative embodiment, right atrial pressure (RAP) can be estimated by correlation to area trace data. For example, paired data of IVC area trace and RAP as measured by pressure catheter, can be used to create a regression model using training data at an individual or population level which can then be used to predict RAP based on an input of the area metrics produced from the sensor area trace, e.g. area mean. Vascular tone, which refers to the degree of constriction experienced by a blood vessel relative to its maximally dilated state, also may be estimated based on area trace data. For example, a change in an extracted feature over time, such as Aₘₐₓ achieved with a maneuver varying between days, may be indicative of changes in IVC tone. Thus correlation to area trace data using a training model can provide IVC tone estimates.

In another embodiment, mean arterial pressure (MAP) can be determined using an external blood pressure cuff and can be combined with the aforementioned cardiac output and RAP (directly measured or estimated as described above) to estimate systemic vascular resistance (SVR) using the following equation [16]:

$$SVR = ( MAP - RAP ) / CO . \qquad [ 16 ]$$

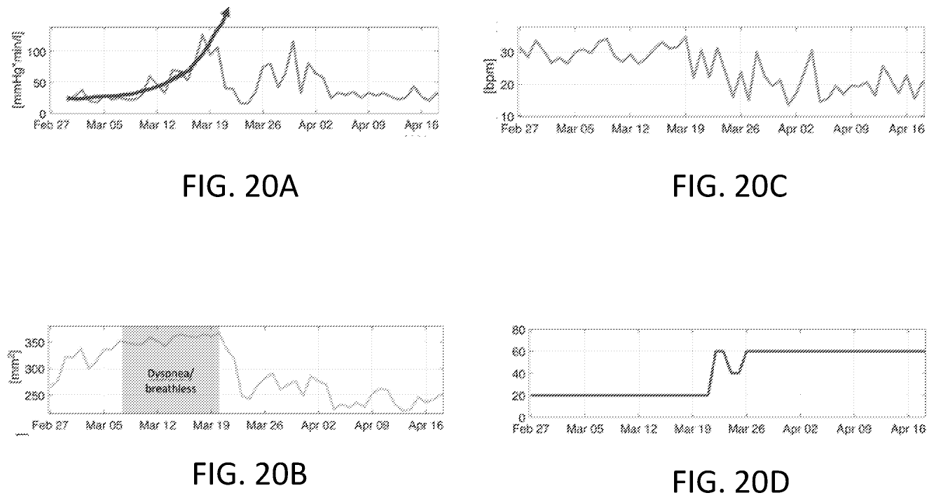

An example of this calculation based on human sensor data is provided in FIG. 20A-D showing trend data as a function of days for a heart failure patient experiencing symptoms (dyspnea). Traces of SVR (systemic vascular resistance), area mean from the sensor, respiration rate from sensor, and daily dose of diuretic (furosemide) are shown. Towards the symptom exacerbation (dyspnea/breathlessness) in mid-March the area increased logarithmically with elevated respiration rate. SVR increased drastically in an exponential shape towards the end of dyspnea. With increase in diuretic dose, area reduced drastically within days and SVR recovered initially to be followed by a brief elevation for several days until medication was ideally titrated to facilitate reduction of patient's respiration rate to normal values.

Systems and methods herein described may be implemented using one or more computing devices and, except where otherwise indicated, may include standard computing components of processors, memory, storage and communications busses, as well as high resolution graphics rendering hardware and software where required. Such components may be configured and programmed by persons of ordinary skill based on the teachings of the present disclosure. Software programing implementing systems and methods described herein may reside on a non-transient computer readable media as a computer program product. Computing devices in general also include cloud-based computing embodiments and computing systems with elements distributed across networks.

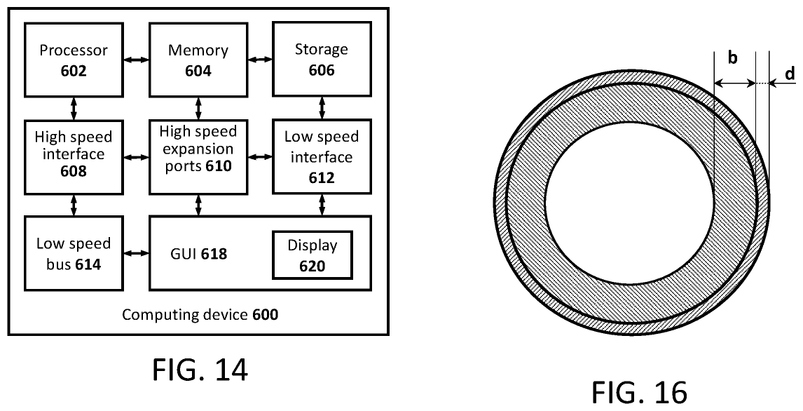

In various alternative embodiments, signal processing, data extraction, diagnostic and treatment-related functions, such as those of trace generator 104, feature detector 106, metrics generator 108, boundary generator 110, index generator 112, decision logic 114 or interface devices 116, including processor 406, as non-limiting examples, may be executed as one or more computing devices, or may be collectively executed in a single or plural computing device. FIG. 14 illustrates one example of such a computing device, wherein computing device 600 includes one or more processors 602, memory 604, storage device 606, high speed interface 608 connecting to memory 604 and high speed expansion ports 610, and a low speed interface 612 connecting to low speed bus 614 and storage device 606. Each of the components 602, 604, 606, 608, 610, and 612, are interconnected using various buses or other suitable connections as indicated in FIG. 8 by arrows connecting components. The processor 602 can process instructions for execution within the computing device 600, including instructions stored in the memory 604 or on the storage device 606 to display graphical information via GUI 618 with display 620, or on an external user interface device, coupled to high speed interface 608. As will be appreciated by persons of ordinary skill, certain described functions, may not require an independent GUI or display. In other implementations, multiple processors and/or multiple buses may be used, as appropriate, along with multiple memories and types of memory. Also, multiple computing devices 600 may be connected, with each device providing portions of the necessary operations (e.g., as a server bank, a group of blade servers, or a multi-processor system).

Memory 604 stores information within the computing device 600. In one implementation, the memory 604 is a computer-readable medium. In one implementation, the memory 604 is a volatile memory unit or units. In another implementation, the memory 604 is a non-volatile memory unit or units.

Storage device 606 is capable of providing mass storage for computing device 600, and may contain information such as timing control, time slice size and/or static color chroma and timing as described hereinabove. In one implementation, storage device 606 is a computer-readable medium. In various different implementations, storage device 606 may be a floppy disk device, a hard disk device, an optical disk device, or a tape device, a flash memory or other similar solid state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer- or machine-readable medium, such as the memory 604, the storage device 606, or memory on processor 602.

High speed interface 608 manages bandwidth-intensive operations for computing device 600, while low speed interface 612 manages lower bandwidth-intensive operations. Such allocation of duties is exemplary only. In one implementation, high speed interface 608 is coupled to memory 604, display 620 (e.g., through a graphics processor or accelerator), and to high-speed expansion ports 610, which may accept various expansion cards (not shown). In the implementation, low speed interface 612 is coupled to storage device 606 and low speed expansion port 614. The low speed expansion port, which may include various communication ports (e.g., USB, Bluetooth, Ethernet, wireless Ethernet) may be coupled to one or more input/output devices as part of GUI 618 or as a further external user interface, such as a keyboard, a pointing device, a scanner, or a networking device such as a switch or router, e.g., through a network adapter.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one out-put device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device separate from video display 620. LED displays are now most common, however older display technologies (e.g., a CRT (cathode ray tube) or LCD (liquid crystal display) monitor) may be used. Other interface devices may include a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feed-back, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of wired or wireless digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other. Processing capacities and capabilities described herein also may be implemented as cloud-based or other network-based processing modules and may also be implemented using a software as a service (“SaaS”) model.

Further alternative embodiments of the present disclosure include methods of creating patient-specific heart failure diagnostic tools. Such methods may comprise monitoring a patient physiological parameter correlated to patient fluid volume, the physiological parameter having a measurable value; instructing the patient to perform a maneuver; recording a change in value of the monitored physiological parameter in response to the patient performing the maneuver; setting the value of the physiological parameter upon the patient performing the maneuver as a maximum value for the patient; determining a minimum value; and setting the maximum value as an upper limit and the minimum value as a lower limit of a standardized scale correlated to a risk of an adverse heart failure event to establish a customized heart failure risk evaluation tool for the patient. With such methodology, measured values of the monitored physiological parameter are comparable to the standardized scale for determination of a level or type of medical intervention.

In another alternative embodiment, a heart failure diagnostic method comprises steps of monitoring a patient physiological parameter correlated to patient fluid volume, the physiological parameter having a measurable value; instructing the patient to perform a maneuver; recording a change in value of the monitored physiological parameter in response to the patient performing the maneuver; setting the value of the physiological parameter upon the patient performing the maneuver as a maximum value for the patient; determining a minimum value; setting the maximum value as an upper limit and the minimum value as a lower limit of a standardized scale correlated to a risk of an adverse heart failure event; comparing measured values of the monitored physiological parameter to the standardized scale; and determining a level or type of medical intervention for the patient based on said comparing to the standardized scale.

The foregoing methods may further comprise collecting IVC area trace data for plurality of patients at a plurality of fluid volume statuses; storing the collected data in a database; analyzing the data to determine curve between max and min values for many different max values; and creating the standardized scale based on the determined curve. In other embodiments, the patient physiological parameter correlated to patient fluid volume comprises an IVC dimension; and the monitoring comprises measuring the IVC dimension. The monitoring may further comprise measuring the IVC dimension with an implanted wireless sensor. In another alternative, the monitoring may comprise measuring the IVC dimension and external ultrasound imaging device. The systems and methods described herein provide unique data and novel data interpretation tools to help clinicians manage heart failure, reduce hospitalizations, better manage patient treatments and improve thus patient outcomes as compared to traditional heart failure management programs.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure or of the inventions as set forth in following claims.

1. An automated heart failure diagnostic device, comprising:
a trace feature detector to identify selected features and at least one of magnitude and timing of the identified features for received periodic vessel area traces representing changes in fluid state of a patient over time, wherein the selected features comprise one or more of interval time per respiration cycle, area magnitude of respiration modulation, interval time per cardiac cycle, area magnitude of cardiac modulation, dominant cardiac peaks, second cardiac peaks, respiration related area reduction, maneuver types and maximum and minimum areas associated with identified maneuvers;
a metrics generator to generate heart function-related parameters for each area trace based on said identified features, magnitudes and timing, said heart function-related parameters comprising one or more of maximum vessel area (Aₘₐₓ), minimum vessel area (Aₘᵢₙ), mean vessel area (Aₘₑₐₙ), heart rate (HR), respiration rate (RR), collapsibility index (CI), collapse (C) and cardiac output (CO);
a boundary generator to generate at least a vessel lower area boundary (LB) for the patient or a vessel upper area boundary (UB) for the patient using said generated heart function-related parameters; and
an index generator to set a patient congestion index based on one or more of said generated heart-function related parameters and at least one of a vessel lower area boundary (LB) or upper area boundary (UB) for the patient, said congestion index indicating patent fluid state on a normalized scale for each said trace period.

2. The device of claim 1, further comprising a trace generator configured to produce the periodic vessel area traces.

3. The device of claim 1, further comprising a decision logic configured to determine at least one of a diagnostic status and treatment protocol for the patient based at least on the congestion index from the index generator.

4. The device of claim 1, wherein the boundary generator sets the lower area boundary (LB) according to one or more of the relationships:
as an average minimum area measured in the IVC using either multiple measurements from the patient or population information from multiple patients;
as equal to (−1/S)·C+Aₘₑₐₙ, wherein (S) is a received reference slope; and
as the minimum vessel area (Aₘᵢₙ) for an area trace associated with a detected maneuver.

5. The device of claim 4, wherein the boundary generator sets the upper area boundary (UB) according to one or more of the relationships:
as an average maximum area measured in the IVC using at least one of multiple measurements from the patient or population information from multiple patients;
as equal to (−1/S)·CI+Aₘₑₐₙ, wherein (S) is a received reference slope; and
as the maximum vessel area (Aₘₐₓ) for an area trace associated with a detected maneuver.

6. The device of claim 1, wherein the index generator sets the congestion index according to one or more of the following relationships:
as equal to (Aₘₐₓ−Aₘᵢₙ)/(Aₘₐₓ−LB)*100%;
as equal to 100*(Aₘₑₐₙ/UB);
as equal to 100*((Aₘₑₐₙ−LB)/(UB−LB)); and
as equal to 100−(Aₘₐₓ−Aₘᵢₙ)/(Aₘₐₓ−LB)*100%.

7. The device of claim 1, wherein the metrics generator is further configure to generate one or more of right atrial pressure (RAP), IVC Tone, or systemic vascular resistance (SVR), wherein:
RAP is predicted based on correlation between received area traces and measured right atrial pressures;
IVC Tone is correlated to area trace maximum area over time using a training model; and
SVR is set as equal to measured mean atrial pressure less RAP divided by collapse (CO).

8. The device of claim 1, further comprising a machine learning based model applying weighting to the congestion index, generated heart function-related parameters and input patient specific information to return a hospitalization probability score for the patient.

9. The device of claim 2, wherein the trace generator comprises at least one of:
an external ultrasound transducer;
an implanted ultrasound sensor;
an implanted resonant circuit coil sensor; or
an implanted sensor including sensing electrodes.

10. A system automatedly determining patient fluid state using periodic vessel area traces, the system comprising:
a trace feature detector to identify selected features of the vessel area traces and at least one of magnitude and timing of the identified features for the vessel area traces;
a metrics generator to generate heart function-related parameters for each area trace based on said identified magnitudes and timing, said heart function-related parameters including at least maximum vessel area (Aₘₐₓ) and minimum vessel area (Aₘᵢₙ); and
an index generator to generate a patient congestion index based on at least said maximum vessel area (Aₘₐₓ), said minimum vessel area (Aₘᵢₙ) and at least one of a vessel lower area boundary (LB) or upper area boundary (UB) for the patient, said congestion index indicating patent fluid state on a normalized scale for each said trace period.

11. The system of claim 10, wherein the selected area trace features further comprise one or more of interval time per respiration cycle, area magnitude of respiration modulation, interval time per cardiac cycle, area magnitude of cardiac modulation, dominant cardiac peaks, second cardiac peaks, respiration related area reduction, maneuver types and maximum and minimum areas associated with identified maneuvers.

12. The system of claim 10, wherein metrics generator generates heart function-related parameters further comprising one or more of mean vessel area (Aₘₑₐₙ), heart rate (HR), respiration rate (RR), collapsibility index (CI), collapse (C) and cardiac output (CO).

13. The system of claim 12, wherein the metrics generator is configured to receive patient specific information and generate the heart function-related parameters based further on received patient specific information.

14. The system of any of claim 13, further comprising a boundary generator to generate at least said lower area boundary (LB) or said upper area boundary (UB) using said generated heart function-related parameters.

15. The system of claim 14, wherein the boundary generator:
receives at least maneuver detection and minimum vessel area (Aₘᵢₙ); and
sets the lower area boundary (LB) as the minimum vessel area (Aₘᵢₙ) for an area trace associated with the maneuver detection.

16. The system of claim 14, wherein the boundary generator:
receives a collapse value (C) and mean area (Aₘₑₐₙ) for an area trace, and a reference slope (S); and
sets the lower area boundary (LB) as equal to (−1/S)·C+Aₘₑₐₙ.

17. The system of claim 14, wherein the boundary generator:
receives minimum area (Aₘᵢₙ) for a selected number of area traces; and
sets the lower area boundary (LB) as an average minimum area measured in the IVC using either multiple measurements from the individual patient or population information from multiple patients.

18. The system of claim 14, wherein the boundary generator:
receives at least maneuver detection and maximum vessel area; and
sets the upper area boundary (UB) as the maximum vessel area for an area trace associated with the maneuver detection.

19. The system of claim 14, wherein the boundary generator:
receives a collapsibility index value (CI) and mean area (Aₘₑₐₙ) for an area trace, and
a reference slope (S); and
sets the upper boundary (UB) as equal to (−1/S)·CI+Aₘₑₐₙ.

20. The system of claim 14, wherein the boundary generator:
receives maximum area (Aₘₐₓ) for a selected number of area traces; and
sets the upper area boundary (UB) as an average maximum area measured in the IVC using at least one of multiple measurements from the patient or population information from multiple patients.

21. The system of claim 10, further comprising a decision logic to determine at least one of a diagnostic status and treatment protocol for the patient based at least on the congestion index from the index generator.

22. The system of claim 21, wherein the decision logic:
generates instructions to signal an alert for user attention and review of risk of hypovolemia in response to a congestion index between about 0-30;
generates instructions to signal a user notification of patient fluid status in a normal range in response to a congestion index between about 30-70; and
generates instructions to signal an alert for user attention and review of risk of hypervolemia in response to a congestion index between about 70-100.

23. The system of claim 22, wherein:
the decision logic additionally receives plural heart function-related parameters from the metrics generator; and
the decision logic comprises a dataset trained model that returns a probability of patient hospitalization based on the Congestion Index and input heart function-related parameters.

24. (canceled)

25. The system of claim 10, further comprising one or more interface devices, wherein:
said one or more interface devices comprise a patient personal device configured to wirelessly communicate with at least said metrics generator and said index generator;
the patient personal device is configured for input of the patient specific information; and
the patient personal device is configured to receive and display the patient congestion index.

26. The system of claim 25, wherein:
said one or more interface devices further comprise a healthcare provider device configured to communicate with the metrics generator, index generator and decision logic;
the health care provider device is configured for input of patient specific information;
the healthcare provider device is configured for input of treatment and diagnostic algorithm changes in the decision logic; and
the health care provider device is configured to receive and display generated heart function related parameters, patent treatment or diagnostic status alerts, and the patient congestion index.

27. The system of claim 10, further comprising a trace generator, wherein the trace generator comprises:
at least one transducer configured to monitor changes in vessel area and produce a sensor signal representative of the monitored changes; and
a processing system configured to receive the sensor signal and produce said periodic area traces based on the sensor signal.

28. The system of claim 27, wherein said at least one transducer comprises an implantable coil configured to produce a variable frequency signal correlated to changes in vessel area when positioned within a vessel.

29. The system of claim 28, wherein said processing system comprises:
a patient wearable antenna;
an energizing circuit communicating with the antenna; and
a signal receiving circuit communicating with the antenna.

30. The system of claim 27, wherein said at least one transducer is configured as a sensor comprising:
an expandable and collapsible variable inductance coil comprising a plurality of adjacent wire strands formed around an open center to allow substantially unimpeded blood flow therethrough, said coil configured and dimensioned to move with the vessel wall in response to expansion and collapse of the vessel; and
a capacitance which together with said variable inductance coil forms a variable inductance resonant circuit having a variable characteristic frequency correlated to the diameter or area of the expandable and collapsible variable inductance coil.

31. (canceled)

32. (canceled)

33. The system of claim 12, wherein the metrics generator:
receives vessel area maximum (Aₘₐₓ) and the vessel area minimum (Aₘᵢₙ); and
sets collapse (C) as equal to Aₘₐₓ−Aₘᵢₙ.

34. The system of claim 12, wherein the metrics generator:
receives collapse (C) and vessel area maximum; and
sets collapsibility index (CI) as equal to C/Aₘₐₓ*100%.

35. (canceled)

36. (canceled)

37. The system of claim 10, wherein the index generator:
receives vessel area maximum (Aₘₐₓ), vessel area minimum (Aₘᵢₙ), and lower area boundary (LB); and
sets the congestion index as equal to (Aₘₐₓ−Aₘᵢₙ)(Aₘₐₓ−LB)*100%.

38. The system of claim 10, wherein the index generator:
receives mean vessel area and an upper boundary; and
sets the congestion index as equal to 100*(Aₘₑₐₙ/UB).

39. The system of wherein the index generator:
receives vessel area maximum (Aₘₐₓ), vessel area minimum (Aₘᵢₙ), upper area boundary (UB) and lower area boundary (LB); and
sets the congestion index as equal to 100*((Aₘₑₐₙ−LB)/(UB−LB)).

40. The system of claim 39, wherein the index generator is further set as equal to 100−(Aₘₐₓ−Aₘᵢₙ)(Aₘₐₓ−LB)*100%.

41. A computer-based method, comprising:
receiving a quiet respiration vessel area trace for a patient within at least one processing device;
receiving patent specific information comprising at least patient weight and patient age within said processing device;
filtering the quiet respiration vessel area trace at said processing device to identify component signals comprising at least a respiration trace, a cardiac trace, and a mean trace;
extracting magnitude and timing features from said traces at said processing device corresponding to at least one or more of area maximum, area minimum, collapse, respiration collapse, cardiac collapse, heart rate and respiration rate;
generating at said processing device heart function-related parameters using executable program instructions defining said heart function-related parameters based on said extracted magnitude and timing features, the heart function-related parameters comprising one or more of respiration rate, cardiac output, heart rate, collapsibility index, collapse, maximum vessel area, minimum vessel area and mean vessel area;
generating a patient congestion index based on one or more of said magnitude features and heart function-related parameters; and
applying weighting to the congestion index, heart function-related parameters and patient specific information using a machine learning based model to return a hospitalization probability score for the patient.

42. (canceled)

43. (canceled)

44. (canceled)

45. A computer-based system comprising one or more processing and memory devices configured and programed to execute the method of claim 41.

46. A method for automatedly determining patient fluid state using periodic vessel area traces, comprising:
receiving a vessel area trace;
identifying selected features and at least one of magnitude and timing of the identified features for the vessel area traces;
generating heart function-related parameters for each area trace based on said identified magnitudes and timing, said heart function-related parameters including measured vessel areas and a vessel area boundary;
generating a patient congestion index representing a relationship between measured vessel area as determined for an area trace and a vessel area boundary for the patient, said congestion index indicating patent fluid state on a normalized scale for each said trace period.

47. The method of claim 46, further comprising generating at least one of a lower area boundary (LB) or an upper area boundary (UB) using said generated heart function-related parameters, and wherein said generating the patient congestion index is based on at least one of said lower area boundary or upper area boundary.

48. The method of claim 47, wherein said generating at least one of a lower area boundary (LB) or an upper area boundary (UB) comprises:
detecting a maneuver in the received area trace;
detecting a minimum vessel area corresponding to the detected maneuver; and
setting the lower area boundary (LB) as said detected minimum vessel area.

49. The method of claim 47, wherein said generating at least one of a lower area boundary (LB) or an upper area boundary (UB) comprises:
receiving a collapse value (C) and mean area (Aₘₑₐₙ) for an area trace;
receiving a reference slope (S); and
setting the lower area boundary (LB) as equal to (−1/S)·C+Aₘₑₐₙ.

50. The method of claim 47, wherein said generating at least one of a lower area boundary (LB) or an upper area boundary (UB) comprises:
receiving a minimum area (Aₘᵢₙ) for a selected number of area traces; and
setting the lower area boundary (LB) as an average minimum sensor size measured in the IVC using either multiple measurements from the individual patient or population information from multiple patients.

51. The method of claim 47, wherein said generating at least one of a lower area boundary (LB) or an upper area boundary (UB) comprises:
detecting a maneuver in the received area trace;
detecting a maximum vessel area corresponding to the detected maneuver; and
setting the upper area boundary (UB) as said detected maximum vessel area.

52. The method of claim 47, wherein said generating at least one of a lower area boundary (LB) or an upper area boundary (UB) comprises:
receiving a collapsibility index value (CI) and mean area (Aₘₑₐₙ) for an area trace;
receiving a reference slope (S); and
setting the upper boundary (UB) as equal to (−1/S)·CI+Aₘₑₐₙ.

53. The method of claim 47, wherein said generating at least one of a lower area boundary (LB) or an upper area boundary (UB) comprises:
receiving a maximum area (Aₘₐₓ) for a selected number of area traces; and
setting the upper area boundary (UB) as an average maximum sensor size measured in the IVC using at least one of multiple measurements from the patient or population information from multiple patients.

54. (canceled)

55. The method of claim 47, further comprising determining a treatment protocol for the patient based at least on the congestion index.

56. The method of claim 47, further comprising:
generating an alert for user attention and review of risk of hypovolemia in response to a Congestion Index between about 0-30;
generating a user notification of patient fluid status in a normal range in response to a Congestion Index between about 30-70; and
generating an alert for user attention and review of risk of hypervolemia in response to a Congestion Index between about 70-100.

57. (canceled)

58. The method of claim 46, further comprising generating periodic vessel area traces for the patient.

59. The method of claim 58, wherein said generating periodic vessel area traces comprises:
monitoring changes in vessel area using a transducing device to produce a sensor signal representative of the monitored changes; and
processing the sensor signal to produce said periodic area traces.

60. The method of claim 59, wherein said monitoring comprises:
receiving the sensor signal from sensor implanted in the IVC configured to produce a variable frequency signal correlated to changes in vessel area.

61. (canceled)

62. (canceled)

63. The method of claim 46, wherein said generating heart function-related parameters comprises-:
receiving a vessel area maximum (Aₘₐₓ) and a vessel area minimum (Aₘᵢₙ); and
setting collapse (C) as equal to Aₘₐₓ−Aₘᵢₙ.

64. The method of claim 46, wherein said generating heart function-related parameters comprises-:
receiving collapse (C) and a vessel area maximum; and
setting collapsibility index (CI) as equal to C/Aₘₐₓ*100%.

65. (canceled)

66. The method of claim 46, wherein said generating a patient congestion index comprises:
receiving a vessel area maximum (Aₘₐₓ), a vessel area minimum (Aₘᵢₙ), and a lower area boundary (LB); and
setting the congestion index as equal to (Aₘₐₓ−Aₘᵢₙ)(Aₘₐₓ−LB)*100%.

67. The method of claim 46, wherein said generating a patient congestion index comprises:
receiving a mean vessel area and an upper boundary; and
setting the congestion index as equal to 100*(Aₘₑₐₙ/UB).

68. The method of claim 46, wherein said generating a patient congestion index comprises:
receiving a vessel area maximum (Aₘₐₓ), a vessel area minimum (Aₘᵢₙ), an upper area boundary (UB) and a lower area boundary (LB); and
setting the congestion index as equal to 100*((Aₘₑₐₙ−LB)/(UB−LB)).

69. (canceled)

70. (canceled)

71. The method of claim 46, wherein said method is a computer-based method with said steps executed in one or more processing devices.
